(19) United States
(12) Patent Application Publication
(10) Pub. No.: US 2020/0155813 A1
(43) Pub. Date: May 21, 2020

(54) CATHETER ASSEMBLY

(71) Applicant: ASAHI INTECC CO., LTD., Aichi (JP)

(72) Inventor: Akihiro MIKI, Seto-shi (JP)

(73) Assignee: ASAHI INTECC CO., LTD., Aichi (JP)

(21) Appl. No.: 16/750,011

(22) Filed: Jan. 23, 2020

Related U.S. Application Data
(63) Continuation of application No. PCT/JP2017/027038, filed on Jul. 26, 2017.

(51) Int. Cl. A61M 25/10; A61M 25/00

(52) U.S. Cl. CPC A61M 25/10; A61M 25/0052; A61M 2025/1061; A61M 2025/0006; A61M 25/0009

(57) ABSTRACT

Disclosed herein is a catheter comprising an outer shaft; and an inner shaft that is disposed inside the outer shaft, wherein the inner shaft includes: a first shaft part that is made of a first resin, a second shaft part that is made of a second resin, harder than the first resin, and provided on a proximal end of the first shaft part, and a large diameter portion that is provided between the first shaft part and the second shaft part so that a proximal end portion of the first shaft part and a distal end portion of the second shaft part overlap and has an outer diameter larger than an outer diameter of the first shaft part and an outer diameter of the second shaft part, and the large diameter portion includes a joint part joining the outer shaft and the inner shaft to each other.

### CROSS REFERENCE TO RELATED APPLICATION

This application is a continuation application of International Application No. PCT/JP2017/027038, filed Jul. 26, 2017. The contents of the International Application are incorporated herein by reference in its entirety.

### TECHNICAL FIELD

The present disclosure relates to a catheter assembly for treatment of a disease or for performing a surgical procedure in, for example, a blood vessel or a digestive organ.

### BACKGROUND

A catheter is a medical device formed from a thin tube made from medical grade materials serving a broad range of therapeutic functions. Catheters typically are inserted in the body to treat diseases or perform a surgical procedure. Catheters may be used for cardiovascular, urological, gastrointestinal, neurovascular, and ophthalmic applications.

A balloon catheter is known as a therapeutic catheter that is inserted into a constricted part formed in a blood vessel or a digestive organ to expand the constricted part. The balloon catheter mainly includes a balloon as an expansion body, an outer shaft joined to the proximal end of the balloon, and an inner shaft disposed inside the balloon and the outer shaft. The inner shaft is used for inserting a guide wire, and an expansion lumen provided between the outer shaft and the inner shaft is used for flowing liquid (such as a contrast medium or physiological saline) for expanding the balloon.

Among such balloon catheters, some kind of balloon catheter is known, in which the outer shaft and the inner shaft are joined to each other at the proximal end of the balloon in order to reduce the axial deviation between the outer shaft and the inner shaft, according to, for example, Japanese Patent Document No. 2013-42841.

However, in the above-mentioned balloon catheter, the inner shaft is greatly curved toward the outer shaft side at the joint part between the outer shaft and the inner shaft. Therefore, when the guide wire is inserted into the inner shaft, the guide wire may be caught at the curved joint part.

### SUMMARY

Embodiments of the present disclosure provide a catheter, comprising an outer shaft; and an inner shaft that is disposed inside the outer shaft, wherein the inner shaft includes: a first shaft part that is made of a first resin, a second shaft part that is made of a second resin, harder than the first resin, and provided on a proximal end of the first shaft part, and a large diameter portion that is provided between the first shaft part and the second shaft part so that a proximal end portion of the first shaft part and a distal end portion of the second shaft part overlap and has an outer diameter larger than an outer diameter of the first shaft part and an outer diameter of the second shaft part, and the large diameter portion includes a joint part joining the outer shaft and the inner shaft to each other.

### BRIEF DESCRIPTION OF DRAWINGS

FIG. 1 illustrates a balloon catheter, according to some embodiments of the present disclosure;

FIG. 2 illustrates an enlarged view of a portion A in FIG. 1, according to some embodiments of the present disclosure;

FIG. 3A is a view illustrating a B-B cross section of FIG. 2, according to some embodiments of the present disclosure;

FIG. 3B is a view illustrating a C-C cross section of FIG. 2, according to some embodiments of the present disclosure;

FIG. 3C is a view illustrating a D-D cross section of FIG. 2, according to some embodiments of the present disclosure;

FIG. 4 illustrates another balloon catheter, according to some embodiments of the present disclosure;

FIG. 5A is a view illustrating an E-E cross section of FIG. 4, according to some embodiments of the present disclosure;

FIG. 5B is a view illustrating an F-F cross section of FIG. 4, according to some embodiments of the present disclosure;

FIG. 5C is a view illustrating a G-G cross section of FIG. 4, according to some embodiments of the present disclosure;

FIG. 6 illustrates yet another balloon catheter, according to some embodiments of the present disclosure;

FIG. 7 illustrates an enlarged view of a portion H in FIG. 6, according to some embodiments of the present disclosure;

FIG. 8A is a view illustrating an I-I cross section of FIG. 7, according to some embodiments of the present disclosure;

FIG. 8B is a view illustrating a J-J cross section of FIG. 7, according to some embodiments of the present disclosure;

FIG. 8C is a view illustrating a K-K cross section of FIG. 7, according to some embodiments of the present disclosure;

FIG. 9 illustrates a portion of another balloon catheter, according to some embodiments of the present disclosure;

FIG. 10 illustrates another balloon catheter, according to some embodiments of the present disclosure; and

FIG. 11 illustrates an enlarged view of a portion L in FIG. 10.

### DETAILED DESCRIPTION

Embodiments of the present disclosure aim at providing a catheter capable of preventing a guide wire from being caught at the joint part between the outer shaft and the inner shaft.

A catheter according to one aspect of the present disclosure for solving the above-described problem may include an outer shaft, and an inner shaft that is disposed inside the outer shaft, in which the inner shaft includes a first shaft part that is made of a first resin, a second shaft part that is made of a second resin, harder than the first resin and provided on a proximal end of the first shaft part, and a large diameter portion that is provided between the first shaft part and the second shaft part so that a proximal end portion of the first shaft part and a distal end portion of the second shaft part overlap and the large diameter portion has an outer diameter larger than an outer diameter of the first shaft part and an outer diameter of the second shaft part, and the large diameter portion includes a joint part joining the outer shaft and the inner shaft to each other.

Although the detailed description below refers primarily to a balloon catheter, those skilled in the art will recognize that the embodiments described herein may be used for other types of catheters.

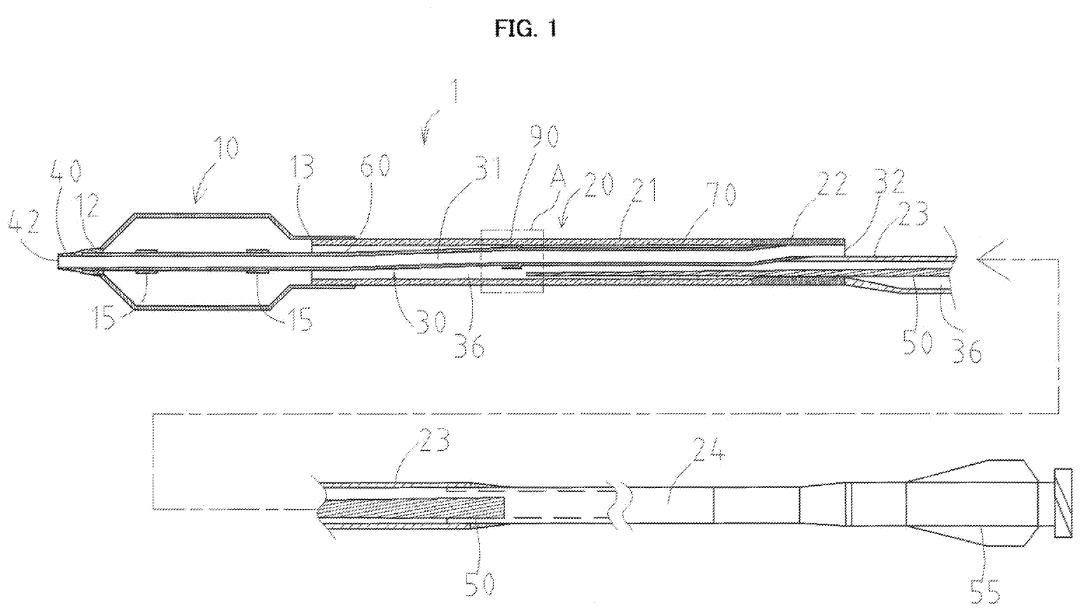

Embodiments of the present disclosure will now be described in detail with reference to FIGS. 1-11.

FIGS. 1, 2 and 3A-3C illustrate a balloon catheter 1, according to some embodiments of the present disclosure. In FIG. 1, the left side of the upper drawing is the distal end (far end) inserted into a body, and the right side of the lower drawing is the proximal end (near end) operated by a technician such as a doctor.

The balloon catheter 1 may be used for expanding and treating a constricted part formed in a blood vessel or a digestive organ, for example. As illustrated in FIG. 1, the balloon catheter 1 may include a balloon 10, an outer shaft 20, an inner shaft 30, a distal tip 40, a core wire 50, and a connector 55.

The balloon 10 may be made of a resin member formed with materials such as a polyether block amide copolymer (nylon-based elastomer resin) and polyurethane resin, and may include a distal end attachment portion 12 on the distal end and a proximal end attachment portion 13 on the proximal end. The distal end attachment portion 12 may be joined to the distal end of the inner shaft 30 through the distal tip 40, and the proximal end attachment portion 13 may be joined to the distal end of the outer shaft 20. In FIG. 1, the distal tip 40 may be provided between the distal end attachment portion 12 and the distal end of the inner shaft 30. However, the embodiment is not limited thereto, and the distal end attachment portion 12 may, for example, be joined directly to the distal end of the inner shaft 30. In addition, the proximal end attachment portion 13 may be joined to the outer periphery of the distal end of the outer shaft 20. However, the embodiment is not limited thereto, and the proximal end attachment portion 13 may, for example, be joined to the inner periphery of the distal end of the outer shaft 20. The balloon 10 may be formed of a single layer or a multi-layer.

The outer shaft 20 may be a tubular member that forms an expansion lumen 36 for supplying liquid such as a contrast medium or physiological saline in order to expand the balloon 10. The outer shaft 20 may include a distal end outer shaft portion 21, a guide wire port portion 22, an intermediate outer shaft portion 23, and a proximal end outer shaft portion 24 in this order from the distal end. The distal outer shaft portion 21 and the intermediate outer shaft portion 23 may be tubes made of resin such as polyamide, polyamide elastomer, polyolefin, polyester, or polyester elastomer. The guide wire port portion 22 may be a portion where the proximal end of the distal end outer shaft portion 21, the distal end of the intermediate outer shaft portion 23, and the proximal end of the inner shaft 30 are joined.

The inner shaft 30 may be inserted into the distal outer shaft portion 21, and the above-described expansion lumen 36 may be formed between the distal end outer shaft portion 21 and the inner shaft 30.

The proximal end outer shaft portion 24 may be a metallic tubular member referred to as a so-called hypotube. The distal end of the proximal end outer shaft portion 24 may be inserted and joined to the proximal end of the intermediate outer shaft portion 23. The connector 55 may be attached to the proximal end of the proximal end outer shaft portion 24. When liquid such as a contrast medium or physiological saline for expanding the balloon 10 is supplied from an indeflator (not illustrated) attachable to the connector 55, the liquid may pass through the expansion lumen 36 and expand the balloon 10. The material of the proximal end outer shaft portion 24 is not particularly limited, and stainless steel (SUS304) or superelastic alloys such as Ni—Ti alloy may be used.

The inner shaft 30 may form a guide wire lumen 31 for inserting a guide wire to the inside. Further, the proximal end of the inner shaft 30 may be joined to the guide wire port portion 22 of the outer shaft 20 to form a proximal end guide wire port 32.

The distal tip 40 may be joined to the distal end of the inner shaft 30. The distal tip 40 may be made of a soft resin. The material is not particularly limited, and polyurethane, polyurethane elastomer, or the like may be used. The distal tip 40 may have a distal end guide wire port 42 at the distal end.

One or more markers 15 having radiopacity may be attached to the inner shaft 30 in the balloon 10 so that the position of the balloon 10 may be observed under irradiation. The number and position of the markers 15 may be appropriately changed depending on the length of the balloon 10.

A core wire 50 may be attached to the inner peripheral surface of the distal end of the proximal end outer shaft portion 24. The core wire 50 may be a tapered metal wire rod having a circular cross section and having a diameter reduced toward the distal end. The material of the core wire 50 is not particularly limited, and stainless steel (SUS304) or superelastic alloys such as Ni—Ti alloy may be used. The core wire 50 may pass through the intermediate outer shaft portion 23 and the guide wire port portion 22, and may extend to the distal end outer shaft portion 21.

The inner shaft 30 may include a first shaft part 60 formed of a flexible first resin, and a second shaft part 70 formed of a second resin harder than the first resin on the proximal end of the first shaft part 60. In an embodiment, a polyether block amide copolymer (nylon elastomer resin) may be used for the first shaft part 60, while polyamide resin may be used for the second shaft part 70. The embodiment is not limited thereto, and may be such that the first shaft part 60 and the second shaft part 70 are formed of the same resin, and the Shore hardness of the first shaft part 60 on the distal end can be lower than the Shore hardness of the second shaft part 70 on the proximal end.

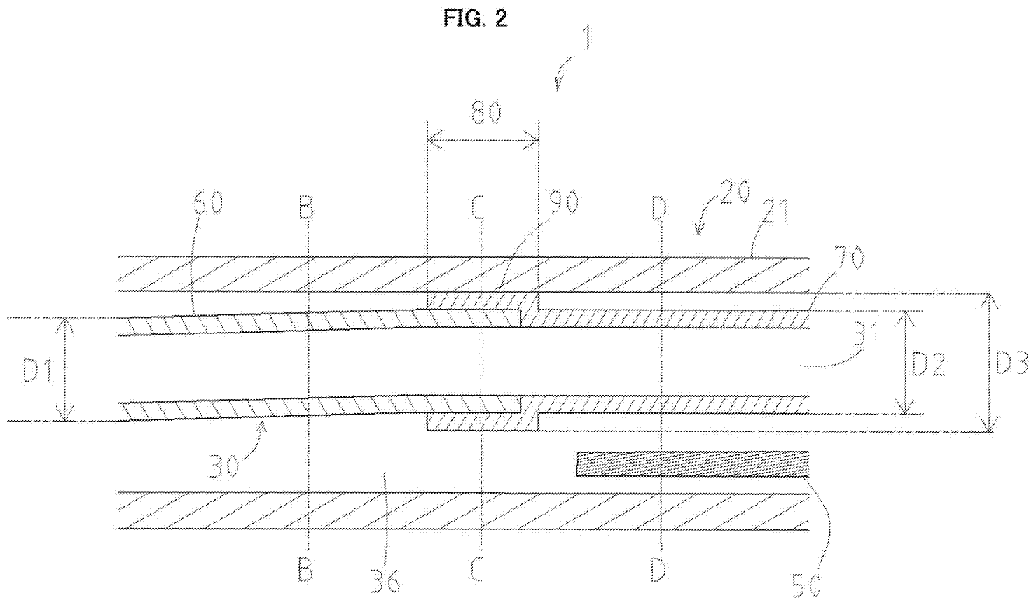

FIG. 2 is an enlarged view of a portion A in FIG. 1, according to some embodiments of the present disclosure. As illustrated in FIG. 2, the distal end portion of the second shaft part 70 may cover the proximal end portion of the first shaft part 60. Therefore, there is provided a large diameter portion 80 that is positioned between the first shaft part 60 and the second shaft part 70 so that the proximal end portion of the first shaft part 60 and the distal end portion of the second shaft part 70 overlap and the large diameter portion 80 has an outer diameter D3 larger than the outer diameter D1 of the first shaft part 60 and the outer diameter D2 of the second shaft part 70.

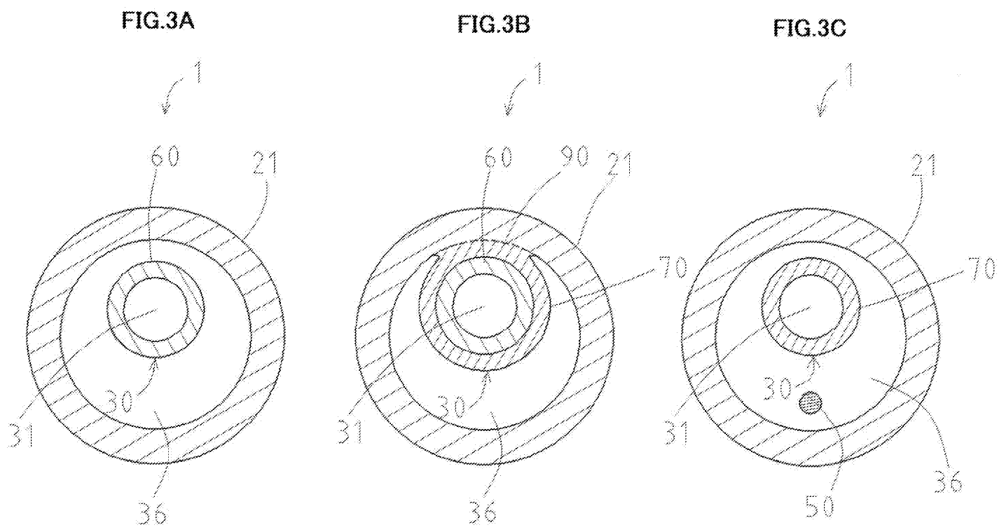

FIG. 3A is a diagram illustrating a B-B cross section of FIG. 2, FIG. 3B is a diagram illustrating a C-C cross section of FIG. 2, and FIG. 3C is a diagram illustrating a D-D cross section of FIG. 2, according to some embodiments of the present disclosure. As illustrated in FIGS. 2 and 3B, the large diameter portion 80 includes a joint part 90 where the outer shaft 20 (distal end outer shaft portion 21) and the inner shaft 30 are joined to each other. In other words, at the joint part 90 of the large diameter portion 80, the inner peripheral surface of the outer shaft 20 is joined to the outer peripheral surface of the distal end portion of the second shaft part 70. As illustrated in FIGS. 2, 3A, and 3C, the outer shaft 20 (the distal end outer shaft portion 21) and the inner shaft 30 are not joined to each other except at the large diameter portion 80.

Thus, in the balloon catheter 1, the outer shaft 20 and the inner shaft 30 are joined to each other at the large diameter portion 80 having the large outer diameter D3. This may reduce the possibility that the inner shaft 30 is curved appreciably to the outer shaft 20 side (paper surface upper side in FIGS. 2 and 3B) in the vicinity of the joint part 90, which consequently may reduce the possibility that the guide wire inserted into the guide wire lumen 31 is caught at the joint part 90.

Further, with the large diameter portion 80 including the joint part 90 joining the outer shaft 20 and the inner shaft 30, it is possible to reduce the possibility that the second shaft portion 70 is pulled out from the first shaft part 60 when the balloon catheter 1 is pulled toward the proximal end. Further, in the large diameter portion 80, the proximal end portion of the first shaft part 60 and the distal end portion of the second shaft part 70 are overlapped, and thereby the thickness of the large diameter portion 80 is increased (see FIGS. 2 and 3B). Therefore, even when the joint part 90 is provided, it is easy to maintain the circularity of the guide wire lumen 31 in the large diameter portion 80, which consequently reduces the possibility that the guide wire is caught at the joint part 90.

In the balloon catheter 1, the second shaft part 70 on the proximal end may be formed of a hard resin, and the first shaft part 60 on the distal end may be formed of a flexible resin. Thus, when the operator pushes the balloon catheter 1 into the distal end, the pushing force of the operator can be easily transmitted to the distal end, thereby allowing the balloon 10 to be easily inserted into a constricted part.

In an embodiment, the joint part 90 may be formed by irradiation with laser light from the outside of the outer shaft 20 in a state where the large diameter portion 80 is drawn toward the outer shaft 20 side (paper surface upper side in FIG. 2 and FIG. 3B). The method for forming the joint part 90 is not particularly limited. For example, the joint part 90 may be formed by joining the outer shaft 20 and the inner shaft 30 (at large diameter portion 80) using an adhesive.

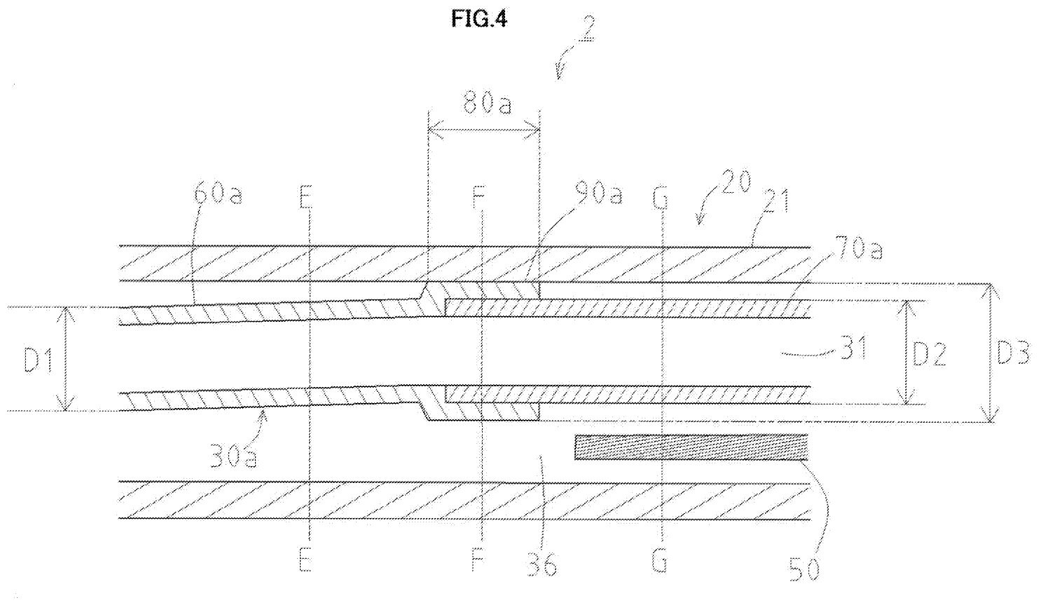

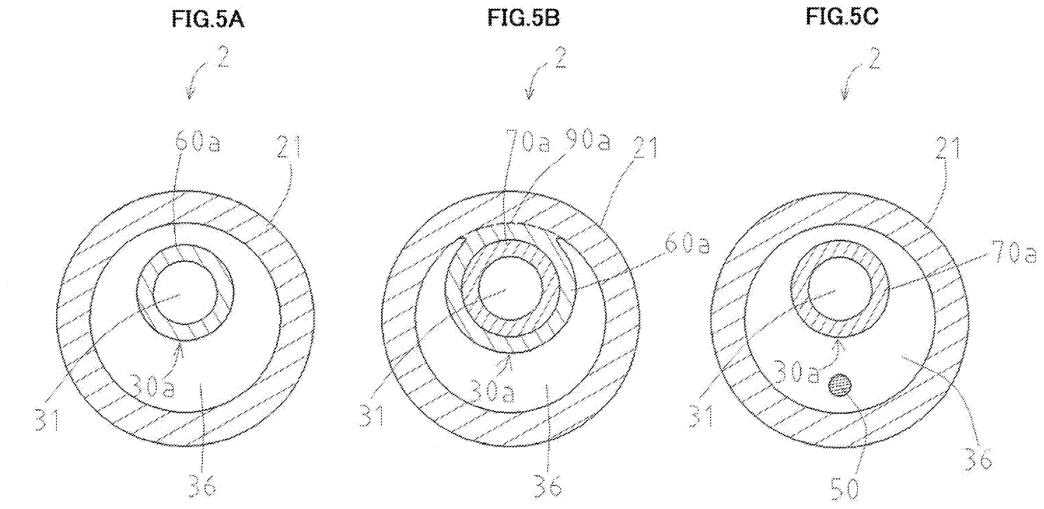

FIGS. 4 and 5A-5C illustrate a balloon catheter 2, according to some embodiments of the present disclosure. Here, the same members as those of the balloon catheter 1 according to the embodiments illustrated in FIG. 2 will be represented with the same reference numerals, and the detailed description thereof will be omitted. Only the differences from the balloon catheter 1 illustrated in FIG. 2 will be described. As illustrated in FIG. 4, an inner shaft 30a may include a first shaft part 60a formed of a flexible first resin and a second shaft portion 70a formed of a second resin harder than the first resin and provided on the proximal end of the first shaft part 60a. The proximal end portion of the first shaft part 60a covers the distal end portion of the second shaft part 70a. Therefore, there is provided a large diameter portion 80a that is formed between the first shaft part 60a and the second shaft part 70a so that the proximal end portion of the first shaft part 60a and the distal end portion of the second shaft part 70a overlap, and thereby the large diameter portion 80a has the outer diameter D3 larger than the outer diameter D1 of the first shaft part 60a and the outer diameter D2 of the second shaft part 70a.

FIG. 5A is a diagram illustrating an E-E cross section of FIG. 4, FIG. 5B is a diagram illustrating an F-F cross section of FIG. 4, and FIG. 5C is a diagram illustrating a G-G cross section of FIG. 4, according to some embodiments of the present disclosure. As illustrated in FIGS. 4 and 5B, the large diameter portion 80a includes a joint part 90a where the outer shaft 20 (distal end outer shaft portion 21) and the inner shaft 30a are joined to each other. In other words, at the joint part 90a of the large diameter portion 80a, the inner peripheral surface of the outer shaft 20 is joined to the outer peripheral surface of the proximal end portion of the first shaft part 60a. As illustrated in FIGS. 4, 5A, and 5C, the outer shaft 20 (the distal end outer shaft portion 21) and the inner shaft 30a are not joined to each other except at the large diameter portion 80a.

Also with this configuration, it is possible to exert the same actions and effects as with the balloon catheter 1 according to some embodiments. In addition, in the balloon catheter 2, with the hard second shaft part 70a existing on the inner side of the flexible first shaft 60a at the joint part 90a between the outer shaft 20 and the inner shaft 30a, the circularity of the guide wire lumen 31 at the joint part 90a is further secured (in other words, the crushing of the inner shaft 30a at the joint part 90a can be further reduced), which consequently reduces the possibility that the guide wire is caught at the joint part 90a.

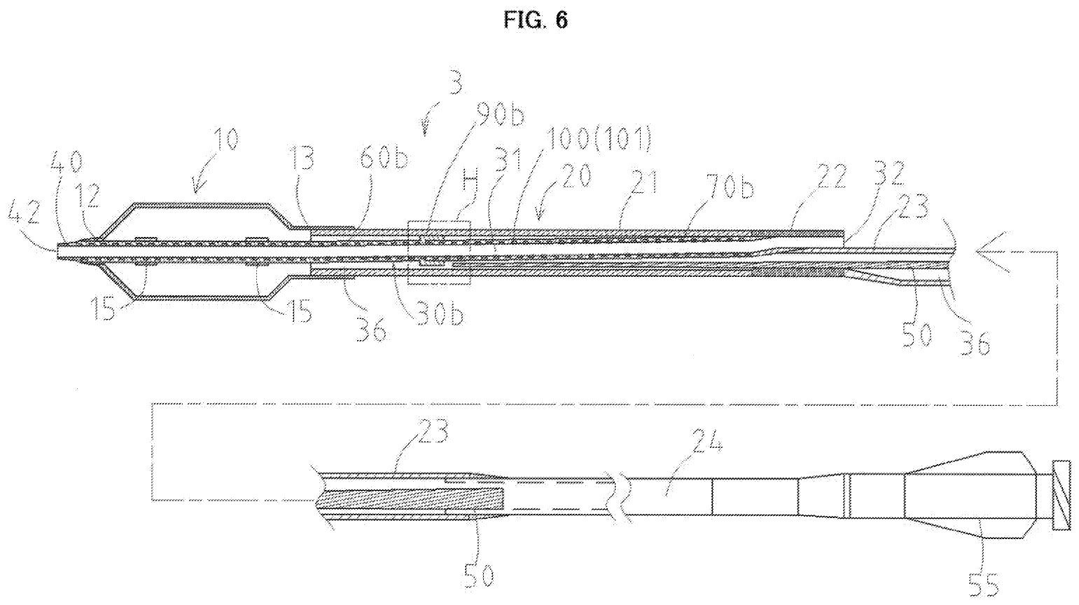

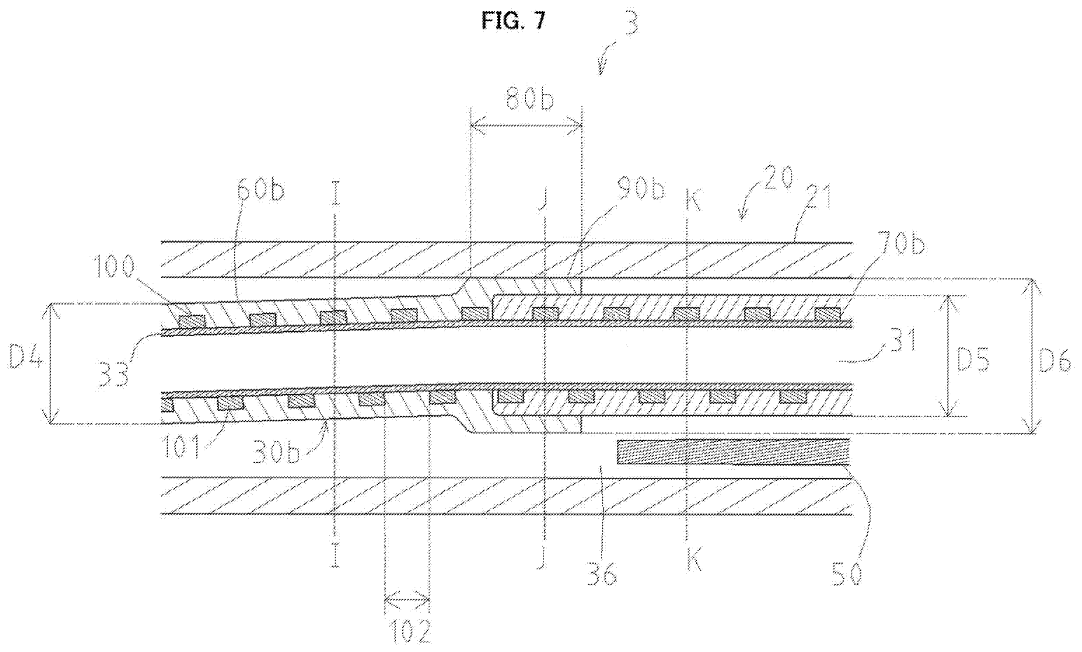

FIGS. 6, 7 and 8A-8C illustrate a balloon catheter 3, according to some embodiments of the present disclosure. Here, the same members as those of the balloon catheter 1 and the balloon catheter 2 will be represented by the same reference numerals, and the detailed description thereof will be omitted. Only the differences from the balloon catheter 1 illustrated in FIG. 1 and the balloon catheter 2 illustrated in FIG. 4 will be described. As illustrated in FIGS. 6 and 7, an inner shaft 30b may include a reinforcing layer 100 that extends from a first shaft part 60b through a large diameter portion 80b to a second shaft part 70b and is wound by strands 101 having a gap 102 between adjacent strands 101. The inner shaft 30b may also include an inner layer 33 that extends from the first shaft part 60b through the large diameter portion 80b to the second shaft part 70b on the inner peripheral side of the reinforcing layer 100.

The material of the strand 101 forming the reinforcement layer 100 is not particularly limited, and stainless steel (SUS304), superelastic alloys such as Ni—Ti alloy, or PEEK (polyether ether ketone) resin may be used. In an embodiment, polytetrafluoroethylene (PTFE) may be used as the inner layer 33. However, the material used for the inner layer 33 is not particularly limited.

As illustrated in FIG. 7, the inner shaft 30b may include the first shaft part 60b that covers the outer periphery of the above-described reinforcing layer 100 and is formed of a flexible first resin and a second shaft part 70b that covers the outer periphery of the reinforcing layer 100 and is formed of a second resin harder than the first resin on the proximal end of the first shaft part 60b. The proximal end portion of the first shaft part 60b may cover the distal end portion of the second shaft part 70b. Therefore, there may be provided the large diameter portion 80b that is formed between the first shaft part 60b and the second shaft part 70b so that the proximal end portion of the first shaft part 60b and the distal end portion of the second shaft part 70b overlap, and thereby the large diameter portion 80b has an outer diameter D6 that is larger than an outer diameter D4 of the first shaft part 60b and an outer diameter D5 of the second shaft part 70b.

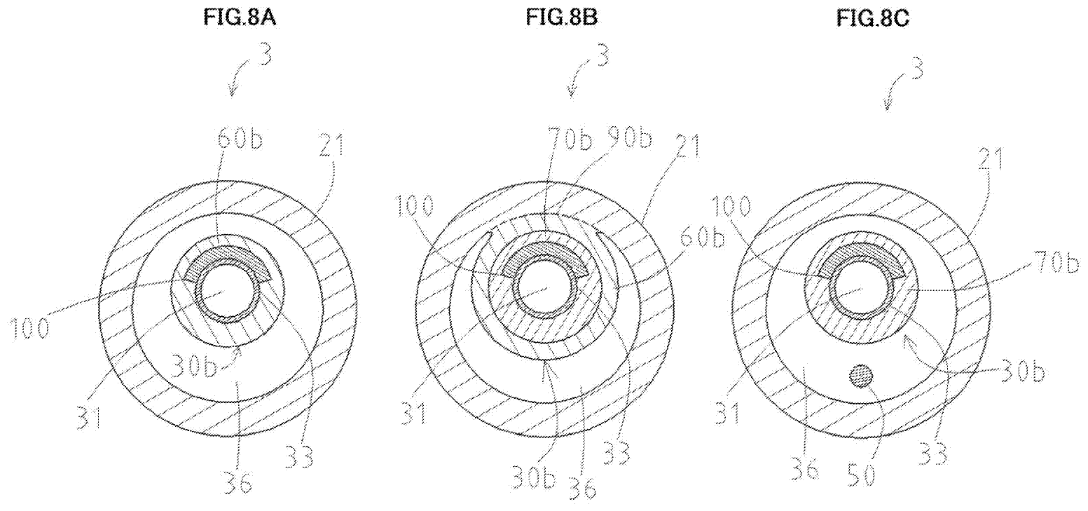

FIG. 8A is a diagram illustrating an I-I cross section in FIG. 7, FIG. 8B is a diagram illustrating a J-J cross section in FIG. 7, and FIG. 8C is a diagram illustrating a K-K cross section of FIG. 7, according to some embodiments of the present disclosure. As illustrated in FIGS. 7 and 8B, the large diameter portion 80b includes a joint part 90b where the outer shaft 20 (distal end outer shaft portion 21) and the inner shaft 30b are joined to each other. In other words, in the joint part 90b of the large diameter portion 80b, the inner peripheral surface of the outer shaft 20 is joined to the outer peripheral surface of the proximal end portion of the first shaft part 60b. As illustrated in FIGS. 7, 8A, and 8C, the outer shaft 20 (the distal end outer shaft portion 21) and the inner shaft 30b are not joined to each other except at the large diameter portion 80b.

Also with this configuration, it may be possible to exert the same actions and effects as the balloon catheter 1 and the balloon catheter 2. In addition, in the balloon catheter 3, with the reinforcing layer 100 existing inside the second shaft part 70b at the joint part 90b between the outer shaft 20 and the inner shaft 30b, the circularity of the guide wire lumen 31 at the joint part 90b is further secured (in other words, the crushing of the inner shaft 30b at the joint part 90b can be further reduced), which consequently reduces the possibility that the guide wire is caught at the joint part 90b.

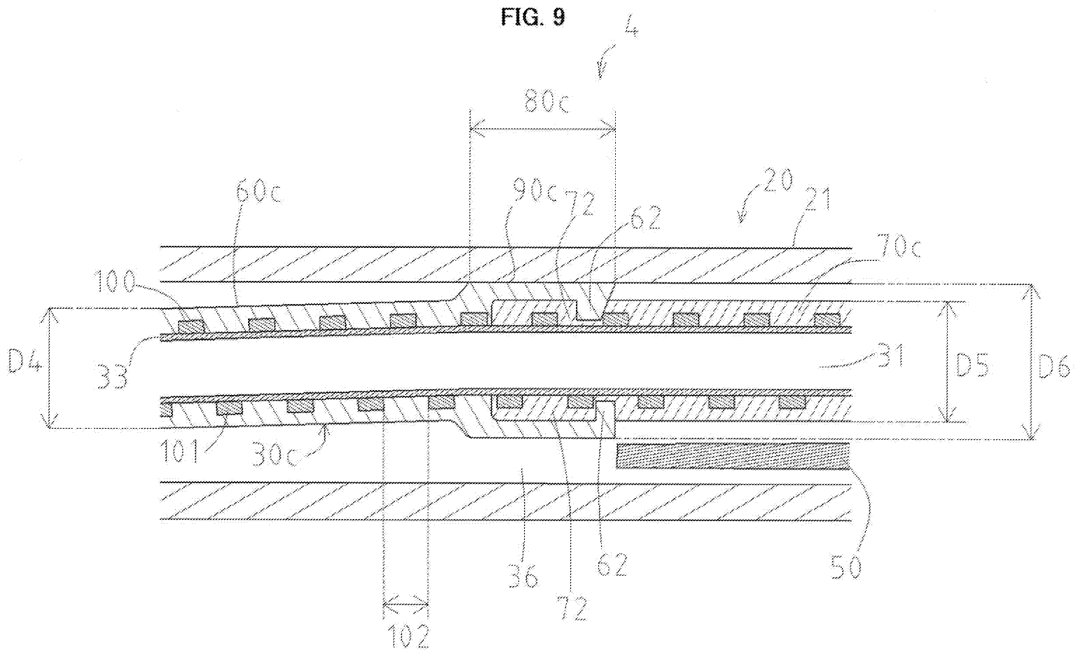

FIG. 9 illustrates a balloon catheter 4, according to some embodiments of the present disclosure. Here, the same members as those of the balloon catheter 3 will be represented with the same reference numerals, and the detailed description thereof will be omitted. Only the differences from the balloon catheter 3 illustrated in FIG. 7 will be described. As illustrated in FIG. 9, an inner shaft 30c may include a first shaft part 60c that covers the outer periphery of the reinforcing layer 100 and is formed of a flexible first resin and a second shaft part 70c that covers the outer periphery of the reinforcing layer 100 and is formed of a second resin harder than the first resin on the proximal end of the first shaft part 60c. A proximal end portion 62 of the first shaft part 60c may cover a distal end portion 72 of the second shaft part 70c. Therefore, between the first shaft part 60c and the second shaft part 70c, there may be provided a large diameter portion 80c having an outer diameter D6 that is larger than the outer diameter D4 of the first shaft part 60c and the outer diameter D5 of the second shaft part 70c at which the proximal end portion 62 of the first shaft part 60c and the distal end portion 72 of the second shaft part 70c overlap.

The large diameter portion 80c may include a joint part 90c where the proximal end portion 62 of the first shaft part 60c is joined to the distal end portion 72 of the second shaft part 70c in a state where the proximal end portion 62 of the first shaft part 60c enters the gap 102 between the strands 101. Therefore, with the anchor effect between the proximal end portion 62 of the first shaft part 60c and the distal end portion 72 of the second shaft part 70c, and the anchor effect between the proximal end portion 62 of the first shaft part 60c and the strand 101 of the reinforcing layer 100, it may be possible to further reduce the possibility that the second shaft part 70c is pulled out from the first shaft part 60c when the balloon catheter 4 is pulled toward the proximal end.

In an embodiment, the joint part 90c may be provided by being irradiated with laser light from the outside of the outer shaft 20 in a state where the large diameter portion 80c is drawn toward the outer shaft 20 side (paper surface upper side in FIG. 9). At this time, the position to be irradiated with the laser beam is adjusted (for example, only the proximal end of the large diameter portion 80c may be irradiated with the laser beam) so that the proximal end portion 62 of the first shaft part 60c is buried in the gap 102 between the strands 101. However, the method of forming the joint part 90c is not limited to this. For example, the joint part 90c may be formed by swaging the proximal end portion 62 of the first shaft part 60c from its outer periphery in a state where the proximal end portion 62 of the first shaft part 60c covers the distal end portion 72 of the second shaft part 70c, so as to form the inner shaft 30c in which the proximal end portion 62 of the first shaft part 60c is buried in the gap 102 between the strands 101, and then joining the inner shaft 30c and the outer shaft 20 by an adhesive.

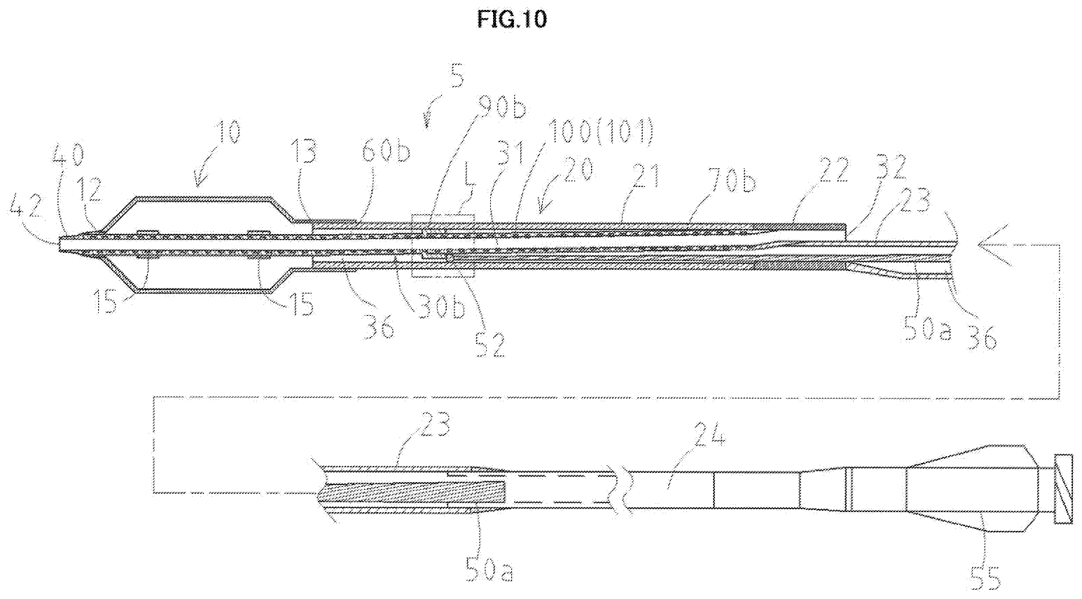

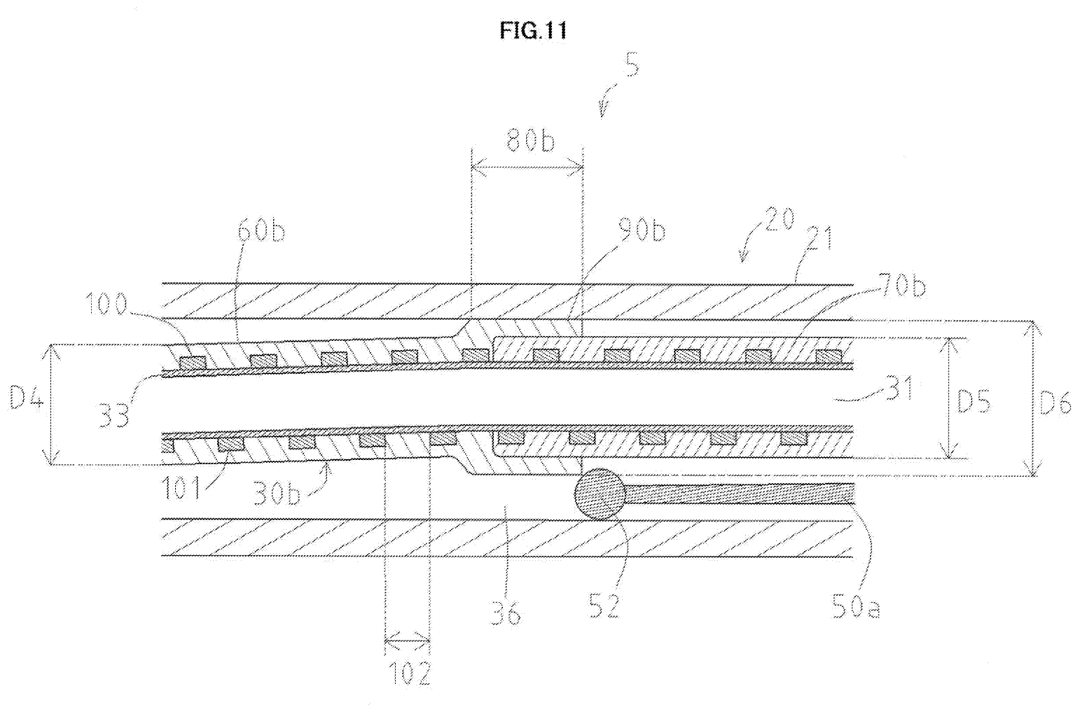

FIGS. 10 and 11 illustrate a balloon catheter 5, according to some embodiments of the present disclosure. Here, the same members as those of the balloon catheter 3 will be represented with the same reference numerals, and the detailed description thereof will be omitted. Only the differences from the balloon catheter 3 illustrated in FIGS. 6 and 7 will be described. As illustrated in FIGS. 10 and 11, a bulging portion 52 may be provided at the distal end of a core wire 50a.

FIG. 11 is an enlarged view of a portion L in FIG. 10, according to some embodiments of the present disclosure. As illustrated in FIG. 11, the bulging portion 52 of the core wire 50a may be in contact with the large diameter portion 80b that is provided so that the proximal end portion of the first shaft part 60b and the distal end portion of the second shaft part 70b overlap and the large diameter portion 80b has an outer diameter D6 larger than the outer diameter D4 of the first shaft part 60b and the outer diameter D5 of the second shaft part 70b. Therefore, in the balloon catheter 5, when the operator pushes the connector 55 in the distal direction, the pushing force may be transmitted from the bulging portion 52 of the core wire 50a to the distal end of the inner shaft 30b through the large diameter portion 80b. At this time, the outer shaft 20 and the large diameter portion 80b are joined to each other at the joint part 90b. Thus, even when the bulging portion 52 of the core wire 50a pushes the large diameter portion 80b toward the distal end, the first shaft part 60b is not separated from the second shaft part 70b.

The first shaft parts 60a, 60b, 60c described in the above-described embodiments may be formed of the same resin material as that of the first shaft part 60 described in the embodiments illustrated in FIGS. 1, 2 and 3A-3C. Similarly, the second shaft parts 70a, 70b, 70c described in the above-described embodiments may be formed of the same resin material as that of the second shaft part 70 described in the embodiments illustrated in FIGS. 1, 2 and 3A-3C.

Further, the bulging portion 52 described in the embodiments illustrated in FIGS. 10 and 11 may be applied to the balloon catheters 1, 2, 3, 4 in the above embodiments.

Further, in the balloon catheters 1, 2, 3, 4, 5, the position of the joint parts 90, 90a, 90b, and 90c is not particularly limited. It may be formed, for example, slightly on the proximal end from the proximal end attachment portion 13 of the balloon 10, or slightly on the distal end from the guide wire port portion 22.

1. A catheter, comprising:
an outer shaft; and
an inner shaft that is disposed inside the outer shaft,
wherein
the inner shaft includes:
a first shaft part that is made of a first resin,
a second shaft part that is made of a second resin, harder than the first resin, and provided on a proximal end of the first shaft part, and
a large diameter portion that is provided between the first shaft part and the second shaft part so that a proximal end portion of the first shaft part and a distal end portion of the second shaft part overlap and has an outer diameter larger than an outer diameter of the first shaft part and an outer diameter of the second shaft part, and
the large diameter portion includes a joint part joining the outer shaft and the inner shaft to each other.

2. The catheter according to claim 1, wherein the large diameter portion is formed so that the proximal end portion of the first shaft part covers the distal end portion of the second shaft part,
the outer shaft is joined to the proximal end portion of the first shaft part at the large diameter portion.

3. The catheter according to claim 1, wherein the large diameter portion is formed so that the distal end portion of the second shaft part covers the proximal end portion of the first shaft part,
the outer shaft is joined to the distal end portion of the second shaft part at the large diameter portion.

4. The catheter according to claim 1, wherein the inner shaft further includes a reinforcing layer that extends from the first shaft part through the large diameter portion to the second shaft part and is wound by strands having a gap between the adjacent strands.

5. The catheter according to claim 4, wherein in the large diameter portion, the proximal end portion of the first shaft part is joined to the distal end portion of the second shaft part in a state of entering the gap between the strands.

6. The catheter according to claim 1, wherein the large diameter portion is formed by irradiation with laser light from outside of the outer shaft.

7. The catheter according to claim 1, wherein the joint part is formed by joining the outer shaft and the inner shaft to each other using an adhesive.

8. A balloon catheter, comprising:
a balloon;
an outer shaft that is joined to a proximal end of the balloon; and
an inner shaft that is disposed inside the outer shaft and joined to a distal end of the balloon, wherein the inner shaft includes:
a first shaft part that is made of a first resin,
a second shaft part that is made of a second resin, harder than the first resin, and provided on a proximal end of the first shaft part, and
a large diameter portion that is provided between the first shaft part and the second shaft part so that a proximal end portion of the first shaft part and a distal end portion of the second shaft part overlap and has an outer diameter larger than an outer diameter of the first shaft part and an outer diameter of the second shaft part, and
the large diameter portion includes a joint part joining the outer shaft and the inner shaft to each other.

9. The balloon catheter according to claim 8, wherein the large diameter portion is formed so that the proximal end portion of the first shaft part covers the distal end portion of the second shaft part,
the outer shaft is joined to the proximal end portion of the first shaft part at the large diameter portion.

10. The balloon catheter according to claim 8, wherein the large diameter portion is formed so that the distal end portion of the second shaft part covers the proximal end portion of the first shaft part,
the outer shaft is joined to the distal end portion of the second shaft part at the large diameter portion.

11. The balloon catheter according to claim 8, wherein the inner shaft further includes a reinforcing layer that extends from the first shaft part through the large diameter portion to the second shaft part and is wound by strands having a gap between the adjacent strands.

12. The balloon catheter according to claim 11, wherein in the large diameter portion, the proximal end portion of the first shaft part is joined to the distal end portion of the second shaft part in a state of entering the gap between the strands.

13. The balloon catheter according to claim 8, wherein the large diameter portion is formed by irradiation with laser light from outside of the outer shaft.

14. The balloon catheter according to claim 8, wherein the joint part is formed by joining the outer shaft and the inner shaft to each other using an adhesive.

15. A balloon catheter, comprising:
a balloon;
an outer shaft that is joined to a proximal end of the balloon; and
an inner shaft that is disposed inside the outer shaft and joined to a distal end of the balloon, wherein an expansion lumen is formed between an outer surface of the inner shaft and an inner surface of the outer shaft;
a core wire disposed in the expansion lumen; and
wherein the inner shaft includes:
a first shaft part that is made of a first resin,
a second shaft part that is made of a second resin, harder than the first resin, and provided on a proximal end of the first shaft part, and
a large diameter portion that is provided between the first shaft part and the second shaft part so that a proximal end portion of the first shaft part and a distal end portion of the second shaft part overlap and has an outer diameter larger than an outer diameter of the first shaft part and an outer diameter of the second shaft part, and
the large diameter portion includes a joint part joining the outer shaft and the inner shaft to each other.

16. The balloon catheter of claim 15, wherein a distal end of the core wire has a bulging portion formed to transmit a pushing force from the core wire to the distal end of the inner shaft through the large diameter portion.

17. The balloon catheter according to claim 15, wherein the large diameter portion is formed so that the proximal end portion of the first shaft part covers the distal end portion of the second shaft part,
the outer shaft is joined to the proximal end portion of the first shaft part at the large diameter portion.

18. The balloon catheter according to claim 15, wherein the large diameter portion is formed so that the distal end portion of the second shaft part covers the proximal end portion of the first shaft part,
the outer shaft is joined to the distal end portion of the second shaft part at the large diameter portion.

19. The balloon catheter according to claim 15, wherein the inner shaft further includes a reinforcing layer that extends from the first shaft part through the large diameter portion to the second shaft part and is wound by strands having a gap between the adjacent strands.

20. The balloon catheter according to claim 19, wherein in the large diameter portion, the proximal end portion of the first shaft part is joined to the distal end portion of the second shaft part in a state of entering the gap between the strands.
